(19) United States
(12) Patent Application Publication
(10) Pub. No.: US 2015/0166457 A1
(43) Pub. Date: Jun. 18, 2015

(54) Method for the Production of Terephthalic Acid and the Derivatives Thereof

(71) Applicant: Oxea GmbH, Oberhausen (DE)

(72) Inventors: Jens Klabunde, Dusseldorf (DE); Heinz Strutz, Moers (DE); Guido D. Frey, Riedstadt (DE)

(73) Assignee: Oxea GmbH, Oberhausen (DE)

(21) Appl. No.: 14/406,890

(22) PCT Filed: Jun. 20, 2013
(86) PCT No.: PCT/EP2013/062853

§ 371 Date: Dec. 10, 2014

(30) Foreign Application Priority Data
Jul. 2, 2012 (DE) 10 2012 105 876.8

(51) Int. Cl. C07C 67/08; C12P 7/62; C07C 5/41; C07C 2/46; C07C 51/265; C07C 2/08

(52) U.S. Cl. CPC C07C 67/08; C07C 51/265; C07C 2/08; C07C 5/41; C07C 2/46; C12P 7/62; C07C 2523/36; C07C 2523/26; C07C 2523/18; C07C 2523/14; C07C 2521/12

(57) ABSTRACT

The present invention relates to a method of producing terephthalic acid derivatives from isobutene via p-xylene. The isobutene is fermentatively produced isobutene, the higher purity of which improves the method and the properties of the produced p-xylene and the terephthalic acid derivatives derived therefrom.

### CLAIM OF PRIORITY

This application is a national phase application of PCT/EP2013/062853 FILED Jun. 20, 2013 which was based on application DE 10 2012 105 876.8 FILED Jul. 2, 2012. The priorities of PCT/EP2013/062853 and DE 10 2012 105 876.8 are hereby claimed and their disclosures incorporated herein by reference.

### TECHNICAL FIELD

The present invention relates to a method for producing terephthalic acid and terephthalic acid derivatives preferably from sources of renewable raw materials.

### BACKGROUND

Terephthalic acid and its derivatives are important industrial compounds that have numerous applications in chemistry. An important application is e.g. the use as a plasticizer, and possibly also as a substitute for phthalic acid derivatives. For the production of terephthalic acid esters e.g. as a substitute product for phthalic acid esters a high isomeric purity of the terephthalic acid component is necessary. After conversion into the corresponding esters particularly relevant phthalate contaminations would not be accepted by end-users.

Processes for the production of terephthalic acid and its derivatives have been known for some time and are described inter alia in Baerns et. al. Technische Chemie, 1st edition, Wiley-VCH, Weinheim, 2006. Usually one starts from p-xylene, which is then oxidized into terephthalic acid and optionally further converted. The xylene itself is, for example, produced from petroleum in refineries, wherein, however, usually isomers are being formed that need to be separated effortfully. Alternatively dimerization processes starting from diisobutene come into consideration, wherein a high isomeric purity of the diisobutene is important, because otherwise xylene isomers are formed, too. Because of the immense importance of terephthalic acid and its derivatives for industrial chemistry, however, it is constantly searched for further improvements with respect to alternative methods and alternative resources of raw materials for the production of terephthalic acid and its derivatives.

The use of renewable raw materials as starting materials for the production of organic chemicals on an industrial scale is becoming increasingly important. On the one hand the resources based on petroleum, natural gas and coal should be conserved and on the other hand with renewable raw materials carbon dioxide is bound in an industrially useable carbon source, which in principal is inexpensive and available in large quantities. Examples for the use of renewable raw materials for the industrial production of organic chemicals include the production of citric acid, 1,3-propanediol, L-lysine, succinic acid, lactic acid, and itaconic acid.

Renewable resources are not yet used for the production of terephthalic acid derivatives. Thus, the task will be to provide an alternative improved method for the production of terephthalic acid derivatives preferably from renewable resources of raw materials. Herein it is of particular importance with regard to the production and use of terephthalic acid derivatives that preferably isomer-free isobutene is used for the production of terephthalic acid derivatives.

### SUMMARY OF INVENTION

The objective of providing an alternative, improved method for the production of terephthalic acid and its derivatives is achieved by a process for producing terephthalic acid and derivatives, comprising the steps of:

a) fermentative preparation of isobutene;

b) conversion of isobutene into p-xylene;

c) oxidation into terephthalic acid; and

d) conversion into terephthalic acid derivatives.

It surprisingly has been found that fermentative produced isobutene has such a high purity with respect to linear butene isomers that the subsequent conversion delivers p-xylene in high purity and yield. This, in turn, results in that in step c) terephthalic acid also in high purity is obtained, and thus possibly finally a terephthalic acid derivative with a high isomeric purity.

In the prior art methods are known in which isobutene is formed biochemically in high purity on a laboratory scale. Thus, however starting from the direct precursor 3-hydroxyisovaleriate (3-hydroxy-3-methylbutyrate), Gogerty, D. S. and Bobik, T. A., 2010, Applied and Environmental Microbiology, pages 8004-8010, investigated the fermentative-enzymatic synthesis of isobutene, wherein according to GC no significant amounts of n-butene isomers were revealed in the valuable product.

The by-product carbon dioxide formed during the fermentation and optionally other inert gases may optionally be removed by suitable separation techniques in a conventional manner. In most embodiments of the invention the conversion of isobutene into p-xylene can be carried out even without further prior purification of the isobutene, thus representing a preferred embodiment of the invention. In this embodiment of the invention the fermentative process of the invention takes advantage of the high selectivity to isobutene as C₄-olefin. On the other side carbon dioxide and other inert gases do not disturb the dimerization of isobutene into diisobutene as an intermediate step of the synthesis of para-xylene from isobutene. In particular cases, however, it may be useful to initially separate carbon dioxide and other inert gases from the isobutene.

### DETAILED DESCRIPTION

The term “fermentative production” of isobutene means particularly that isobutene is derived either
by means of microorganisms, preferably from renewable raw materials; and/or
by a cell-free enzymatic method, also preferably from renewable raw materials.

Isobutene is—as far as is known—not a natural product in the sense that it is formed in metabolic processes in organisms in such amounts that an industrial use seems appropriate. However, isobutene is produced in very small amounts from naturally occurring microorganisms (U.S. Pat. No. 4,698,304; Fukuda, H. et al., 1984, From Agricultural and Biological Chemistry (1984), 48(6), pp. 1679-82). Thus, in the previously known embodiments of the invention, the fermentative preparation of isobutene is done by means of modified, non-natural microorganisms and the corresponding modified enzymes, respectively. Such microorganisms are disclosed in US2011165644 (1l), wherein in Example 13 the synthesis of isobutene from glucose in suitable microorganisms is discussed. In WO2012052427 and WO2011032934 further enzymatic reactions are described, which describe the formation of isobutene as a series of sequential enzymatic syntheses of

I) acetone into 3-hydroxyisovaleriate and

II) 3-hydroxyisovaleriate into isobutene and carbon dioxide.

The enzymatically catalyzed decomposition of 3-hydroxyisovaleriate into isobutene and carbon dioxide is also discussed in Gogerty, D. S. and Bobik, T. A., 2010, Applied and Environmental Microbiology, pages 8004-8010. Here, according to GC, no significant amounts of n-butene isomers were revealed in the valuable product. Even in aqueous, non-enzymatically catalyzed systems one observes a spontaneous separation of carbon dioxide from 3-hydroxyisovaleriate under formation of isobutene, which further reacts with the present water in a balance reaction into tert-butanol (Pressman, D. and Lucas, H. J., 1940, Journal of the American Chemical Society, pages 2069-2081).

If this sequence of enzymatic syntheses described in I and II is included in a suitable microbial host organism which is capable of synthesizing acetone from metabolic precursors or to transport externally supplied acetone by means of a passive or active transport through the cell wall into the cell, by means of a non-natural microorganism derived in such a manner isobutene can be produced by a fermentative process with a good yield. Microorganisms that synthesize acetone from different carbohydrates have long been known and are described inter alia in Jones, T. D. and Woods, D. R., 1986, Microb. Reviews, pages 484-524. Taylor, D. G. et al., 1980, Journal of General Microbiology, 118, pages 159-170, describe microorganisms that use acetone as a sole carbon source and, thus, are able to transport acetone across the cell wall into the cell.

Another possible metabolic pathway proceeds via the reaction sequence:

I) pyruvate into 2-acetolactate;

II) 2-acetolactate into 2,3-dihydroxyisovaleriate;

III) 2,3-dihydroxyisovaleriate into 2-oxoisovaleriate;

IV) 2-oxoisovaleriate into isobutyraldehyde;

V) isobutyraldehyde into isobutanol; and

VI) isobutanol into isobutene

and is described inter alia in WO2011076689 and WO2011076691.

According to a preferred embodiment of the invention no purification of the isobutene is carried out between step a) and b), in particular no purification to remove linear butene isomers and possibly inert gases such as carbon dioxide and/or nitrogen. Herein “purification” means in particular (but not limited thereto) the following methods:
Distillation processes (which, however, are complicated by the fact that the separation of linear butene isomers occurring in the overall process requires a lot of effort, since the boiling points of the isomers are very close to each other, see Kirk-Othmer, Encyclopedia of Chemical Technology, 3rd edition, 1978, vol. 4, John Wiley & Sons Inc., pp. 358-360).
Purification or separation methods in which isobutene is separated due to the increased chemical reactivity by means of a chemical reaction, and then is converted back into isobutene. This includes methods such as reversible proton-catalyzed water addition to the tert-butanol or the methanol addition to methyl-tert-butyl ether (see EP1489062). From these adducts then isobutene is recovered by a reverse reaction (see Weissermel, Arpe, Industrielle Organische Chemie, VCH Verlagsgesellschaft, 3rd edition, 1988, pp. 74-79).

Purification or separation methods in which isobutene is separated from linear butene isomers due to the compact spatial molecular structure by means of suitable physical size exclusion methods, for example, by means of molecular sieves having an appropriate pore size, (see WO2012040859, Weissermel, Arpe, Industrielle Organische Chemie, VCH Verlagsgesellschaft, 3rd edition, 1988, p. 74).
Purification and separation methods suitable for the removal of carbon dioxide.

According to a preferred embodiment of the invention the isobutene is derived in step a) from trisaccharides, disaccharides, monosaccharides, acetone or mixtures thereof. The tri- and disaccharides used are in particular raffinose, cellobiose, lactose, isomaltose, maltose and sucrose. The monosaccharides used are in particular D-glucose, D-fructose, D-galactose, D-mannose, DL-arabinose and DL-xylose. Herein the tri-, di- and monosaccharides inter alia originate (but not limited thereto)
from the digestion and the depolymerization of cellulose and hemicellulose using appropriate methods;
directly from plants with high sugar content such as sugar beet, sugar cane, palm sugar, maple sugar, sorghum, silver date palm, honey palm, palmyra palm and agaves by means of extraction;
from the depolymerization of plant starch by hydrolysis;
from the depolymerization of animal glycogen by hydrolysis;
directly from milk obtained from the dairy industry.

In a further preferred embodiment of the invention exclusively renewable raw materials are used for the fermentative production of isobutene. If desired, the origin of the carbon atoms derived from sources of renewable raw materials can be determined by the test method described in ASTM D6866. Herein the ratio of C¹⁴ to C¹² carbon isotopes is determined and compared with the isotopic ratio of a reference substance, the carbon atoms of which originate in 100% from sources of renewable raw materials. This test method is also known in modified form as radiocarbon method and is described among others in Olsson, I. U., 1991, Euro Courses: Advanced Scientific Techniques, volume 1, Issue Sci. Dating Methods, pages 15-35.

According to a preferred embodiment of the invention the fermentation process is carried out at temperatures of ≧20° C. to ≦45° C. and under atmospheric pressure, wherein isobutene is released as a gaseous product. This embodiment has the advantage that the thus obtained isobutene can be used again directly or after separation of inert gases.

Alternatively the fermentation process is carried out at temperatures of ≧20° C. to ≦45° C. and under a pressure between 1 to 30 bar in accordance with a likewise preferred embodiment of the invention. In this case, isobutene can be obtained as a liquid compound and be separated directly by phase separation from the fermentation medium. In this preferred embodiment the separation of inert gases can be considerably facilitated.

The conversion of isobutene into p-xylene may preferably be implemented in two ways:

1) direct conversion of isobutene into p-xylene; and/or

2) conversion of isobutene into diisobutene, which is then converted into p-xylene.

The two reaction paths, which likewise represent preferred embodiments of the present invention will be described in more detail below:

1) Direct conversion of isobutene into p-xylene

According to a preferred embodiment of the invention, the reaction step b) is such that the isobutene produced fermentatively in accordance with step a) is converted in a reaction step into para-xylene. In this conversion, also known as cyclodimerization, dehydrated amorphous silica gels treated with aluminum hydride (U.S. Pat. No. 4,384,154), bismuth, lead and antimony oxides (U.S. Pat. No. 3,644,550 and U.S. Pat. No. 3,830,866), chromium oxide deposited on alumina (U.S. Pat. No. 3,836,603) or rhenium or rhenium oxide deposited on a neutral or weakly acidic support material (U.S. Pat. No. 4,229,320) can be used as catalysts.

According to a preferred embodiment of this aspect of the invention no purification of the isobutene is carried out between steps a) and b) because the isobutene resulting from step a) is so pure that the cyclodimerization reaction is characterized by a high selectivity to para-xylene.

2) Conversion via diisobutene as an intermediate product

According to a further, likewise preferred embodiment of the invention, the reaction step b) is such that the isobutene produced fermentatively in accordance with step a) is first dimerized to diisobutene in a step b1), which subsequently is further converted in a step b2) into p-xylene.

The term “diisobutene” means, as already described, 2,4,4-trimethyl-1-pentene, 2,4,4-trimethyl-2-pentene as the main components and any mixtures of these two compounds.

According to a preferred embodiment, step b1) is carried out under acid catalysis. Herein, for example, sulfuric acid or acidic ion exchangers come into consideration, as are described inter alia in Weissermel, Arpe, Industrielle Organische Chemie, VCH Verlagsgesellschaft, 3rd edition, 1988, p 77; Hydrocarbon Processing, April 1973, pp. 171-173. Alternatively, the methods described in US 2004/0054246, U.S. Pat. No. 4,100,220 (A), U.S. Pat. No. 4,447,668 (A) and U.S. Pat. No. 5,877,372 (A) can be used.

According to one embodiment of the invention no purification of the diisobutene is carried out between the steps b1) and b2), in particular no purification for removing higher isobutene oligomers and optionally inert gases such as carbon dioxide and/or nitrogen.

Alternatively and preferably the method comprises a further step b1 (i)), which is carried out after step b1):
b1 (i)) Purification of diisobutene, preferably by distillation,

Step b1 (i)) is preferably carried out such that the volatile components which were not converted are separated from the diisobutene and the diisobutene obtained is purified by distillation from the triisobutene and higher isobutene oligomers which may be formed in small quantities. The thus obtained tri-isobutene and the thus obtained higher isobutene oligomers may also be refined into valuable secondary products.

Step b2) is preferably carried out in a dehydrocyclization reaction. Such reactions and conditions for their implementation are described in particular in WO 2011044243.

Step b2) is preferably carried out in the presence of a catalyst. Herein the catalysts are, without limitation, selected from the group consisting of bismuth, lead, and antimony-containing catalysts, platinum catalysts, particularly platinum deposited on zeolite, chromium catalysts, especially chromium oxide deposited on alumina, and mixtures thereof.

Step c) is preferably carried out in a liquid phase oxidation. A preferred oxidizing agent is oxygen. Such processes are inter alia known from A. K. Suresh, Ind. Eng. Chem. Res. 2000, 39, pp. 3958-3997; alternatively, also the methods known from U.S. Pat. No. 2,813,119, U.S. Pat. No. 3,513,193, U.S. Pat. No. 3,887,612, U.S. Pat. No. 3,850,981, U.S. Pat. No. 4,096,340, U.S. Pat. No. 4,241,220 U.S. Pat. No. 4,329,493, U.S. Pat. No. 4,342,876, U.S. Pat. No. 4,642,369 and U.S. Pat. No. 4,908,471 can be used.

Step d) particularly preferably comprises esterification, either mono- or diesterification. Herein mono- or diesters with aliphatic monoalkanols having 1 to 11 carbon atoms are preferred.

According to a preferred embodiment of the invention, the alcohols used for this purpose are obtained from renewable raw materials. Herein particularly alcohols can be used, whose production methods are described hereinafter:

The fermentative production of straight-chain aliphatic mono-alcohols having 1 to 4 carbon atoms such as methanol, ethanol, n-propanol and n-butanol is known from the prior art and is implemented on an industrial scale since decades. The fermentative production of branched-chain isopropanol and isobutanol is also known from the prior art (Sakuragi, H., Journal of Biomedicine and Biotechnology, 2011, pages 1-11). The synthesis of alpha-branched-chain aliphatic mono-alcohols from straight-chain aliphatic mono-alcohols according to the Guerbet reaction is a method known from the prior art (Ullmann's Encyclopedia of Chemical Technology, 5th edition, John Wiley & Sons, 1985, vol. 17, page 287). For example, the condensation reaction of two n-butanol molecules into 2-ethylhexanol according to the Guerbet reaction is described in Matsu-ura, T. Journal of Organic Chemistry 2006, 71 (21), pages 8306-8308. The carbon incorporated in the resulting alpha-branched-chain aliphatic mono-alcohol is derived by 100% from renewable raw materials, if the aliphatic mono-alcohols used originate from fermentation processes that exclusively use sources of renewable raw materials.

The preparation of 2-ethylhexanol can also be implemented via the partial oxidation of n-butanol produced from sources of renewable raw materials into n-butyraldehyde. By means of a subsequent aldol condensation reaction of two n-butyraldehyde molecules into 2-ethyl-3-hydroxy-hexanal, followed by dehydration and catalytic hydrogenation, 2-ethylhexanol can be obtained, wherein all carbon atoms arise to 100% from renewable raw materials. The catalytic partial oxidation of n-butanol into n-butyraldehyde is a method known in the prior art and is, for example, described in Requies, J., Catalysis Letters 2012, 142 (4), pages 417-426.

The esterification (step d) can be carried out with stoichiometric amounts of terephthalic acid and an aliphatic mono-alcohol. However, preferably terephthalic acid is allowed to react with an excess of mono-alcohol which generally is the lower boiling component and which may be separated in a simple way in the subsequent processing of the crude ester by distillation. The aliphatic mono-alcohol is used in a molar excess of 10 to 50%, preferably 20-40% per mole of the acid group of the terephthalic acid to be esterified.

The reaction water formed preferably is distilled off together with the excess mono-alcohol from the reaction vessel during the esterification reaction and supplied to a downstream phase separator, in which mono-alcohol and water separate according to their solubility properties. Possibly the mono-alcohol used together with water also forms an azeotrope under the reaction conditions and is capable to remove the reaction water as an entraining agent. From the accumulated water the course of the reaction can be tracked. The separated water is removed from the process while the mono-alcohol from the phase separator flows back into the reaction vessel. If necessary, an azeotrope former, i.e. a further organic solvent, such as hexane, 1-hexene, cyclohexane, toluene, xylene or xylene isomer mixtures, can be added. The azeotrope former can already be added at the beginning of the esterification reaction or after reaching higher temperatures. If the theoretically expected amount of water is reached or the acid number determined, for example, according to ASTM D 974, has come below a specified level, the reaction is usually completed by allowing the reaction mixture to cool. The esterification of terephthalic acid can be effected at atmospheric pressure, under reduced pressure or at elevated pressure such as to raise the esterification temperature.

Preferred catalysts for the esterification of terephthalic acid with the mono-alcohol are Lewis acids containing at least one element of groups 4 to 14 of the periodic table of the elements, and they can be used in solid or liquid form. The term “Lewis acid” in the sense of the invention generally means the accepted definition of such elements or compounds which have an electron gap, such as described in Römpp's Chemie-Lexikon, 8th edition, Franck'sche Verlagshandlung 1983, volume 3, H-L. Particularly suitable Lewis acids that can be used as catalysts in the esterification reaction include titanium, zirconium, iron, zinc, boron, aluminum or tin, which are used as an element in a finely distributed form or preferably in the form of compounds. Suitable compounds are, for example, tin (II) oxide, tin (IV) oxide, tin carboxylates, such as tin (II)-2-ethylhexanoate, tin (II) oxalate, tin (II) acetate or tin (IV) acetate, tin (IV) alkoxides such as tetra(methyl) stannate, tetra(ethyl) stannate, tetra(propyl) stannate, tetra(isopropyl) stannate or tetra(isobutyl) stannate or organotin compounds such butyltin maleate or dibutyltin dilaurate.

Suitable titanium compounds include alkoxides such as tetra(methyl) orthotitanate, tetra(ethyl) orthotitanate, tetra(propyl) orthotitanate, tetra(isopropyl) orthotitanate, tetra(butyl) orthotitanate, tetra(isobutyl) orthotitanate, tetra(pentyl) orthotitanate or tetra(2-ethylhexyl) orthotitanate; acylates such as hydroxytitanium acetate, hydroxytitanium butyrat or hydroxytitanium pentanoate or chelates such as tetraethylen glycoltitanate or tetrapropylen glycoltitanate. Even the corresponding zirconium compounds can be used with success, such as tetra(methyl) orthozirconate, tetra(ethyl) orthozirconate, tetra(propyl) orthozirconate, tetra(isopropyl) orthozirconate, tetra(butyl) orthozirconate, tetra(isobutyl) orthozirconate, tetra(pentyl) orthozirconate or tetra(2-ethylhexyl) orthozirkonat.

Likewise suitable are boric acid and boric acid ester such as trimethyl borate, triethyl borate, boric acid tripropyl ester, boric acid triisopropyl ester, tributyl borate or boric acid triisobutyl ester.

Likewise suitable are aluminum oxide, aluminum hydroxide, aluminum carboxylates such as aluminum acetate or aluminum stearate, or aluminum alkoxides such as aluminum tri-butylate, aluminum tri-sec-butylate, aluminum tri-tert-butylate or aluminum tri-isopropylate.

Zinc oxide, zinc sulfate and zinc carboxylates, such as zinc acetate dihydrate or zinc stearate, and iron (II) acetate or iron (III) hydroxide-oxide may also be used as catalysts. The catalyst may be added to the reaction mixture already at the beginning or later in compliance with safety measures at an elevated temperature if, for example, the separation of the reaction water has commenced. The catalyst may also be added in portions.

The amount of the added esterification catalyst is 1×10⁻⁵ to 20 mol-%, preferably 0.01 to 5 mol-%, particularly 0.01 to 2 mol-%, with respect to the starting compound added in a deficit amount, suitably with respect to the terephthalic acid. At higher amounts of catalyst amounts depending on the application cleavage reactions of the terephthalic acid ester are to be expected.

The esterification catalyst may be added in liquid or solid form. Solid catalysts such as tin (II) oxide, zinc oxide or iron (III) hydroxide oxide are preferably filtered off after completion of the esterification reaction before the crude terephthalic acid ester is subjected to the further processing. If the esterification catalysts are added as liquid compounds such as tetra(iso-propyl) orthotitanate or tetra(butyl) orthotitanate, which are still present in a solved form in the reaction mixture after completion of the esterification reaction, these compounds are preferably transferred into precipitates which are well filterable in the course of the reprocessing during the steam treatment.

In a particular embodiment of the method according to the invention the esterification is carried out in the presence of an adsorbent. Herein porous, large-area solid materials are used that are commonly used in chemical practice, both in the laboratory and in technical plants. Examples of such materials are high surface-area poly-silicic acids such as silica gels (silica xerogels), silicagel, kieselguhr, high surface-area aluminas and alumina hydrates, mineral materials such as clays or carbonates, or activated carbon. Activated carbon has proved particularly suitable. In general, the adsorbent is suspended finely divided in the reaction solution, which is moved by vigorous agitation or by introducing an inert gas. Thus, an intimate contact is achieved between the liquid phase and the adsorbent. The amount of the adsorbent can substantially be set arbitrarily and thus according to the individual requirements. Based on 100 parts by weight of the liquid reaction formulation it has been proven appropriate to use 0.1 to 5, preferably 0.1 to 1.5 parts by weight of the adsorbent.

The resulting reaction mixture obtained after the conversion beside the terephthalic acid ester as the desired reaction product mostly contains possibly non-converted starting materials, in particular an excess of aliphatic mono-alcohols, provided that according to the preferred embodiment of the method according to the invention an excess of mono-alcohol is used. Usually, initially non-converted starting compounds present in excess are distilled suitably by applying a reduced pressure.

Subsequently the crude ester is subjected to a treatment with steam which in a simple form can be done, for example, by introducing steam into the crude product. An advantage of the steam treatment is that in its course catalyst which is still present is destroyed and is transferred into well-filterable hydrolysis products. If the esterification reaction is conducted in the presence of an adsorbent, the adsorbent already present facilitates the precipitation of the catalyst reaction products. Otherwise, it may prove advantageous to add the adsorbent at the beginning of the steam treatment. The presence of an adsorbent during the steam treatment also has a beneficial effect on the color and the color stability of the terephthalic acid ester. It is also possible to filter off the adsorbent after completion of the esterification reaction and the separation of excess starting compounds, i.e. prior to the steam distillation.

The steam treatment is generally carried out at atmospheric pressure, although the use of a slight negative pressure preferably up to 400 hPa is not excluded. The steam treatment is generally carried out at temperatures from 100 to 250° C., preferably from 150 to 220° C. and especially from 170 to 200° C., and also depends on the physical properties of each terephthalic acid ester to be produced.

In the process step of the steam treatment it proves appropriate to proceed as cautiously as possible during the heating period until the operation temperature is reached in order to heat the crude ester to the required temperature for the steam treatment.

If appropriate, after the steam treatment a solid alkaline material, for example, basic silica, basic aluminum oxide or sodium carbonate, sodium hydrogen carbonate, calcium carbonate, or sodium hydroxide in solid form as well as alkaline minerals are added to further reduce the neutralization number of the terephthalic acid ester.

Subsequently to the steam treatment, possibly after filtration of the adsorbent, the optionally added solid alkaline substances and other solids arised, the terephthalic acid ester is dried, for example, by passing an inert gas at an elevated temperature through the product. Concurrently a vacuum can be applied at an elevated temperature and, where appropriate, an inert gas can be passed through the product. Even without the action of an inert gas the process can be carried out at an elevated temperature or only at a lower pressure.

In general, temperatures in the range of 80 to 250° C., preferably 100 to 180° C., and pressures from 0.2 to 500 hPa, preferably from 1 to 200 hPa and more preferred from 1 to 20 hPa are used during the process. Subsequently the crude ester, if not yet done, is filtered, in order to remove solids, solid alkaline substances, if added, the hydrolysis products of the catalyst and the adsorbent, if added during the esterification step or prior to the steam treatment. The filtration is preferably carried out in conventional filtration apparatuses at normal temperature or at temperatures up to 120° C. The filtration can be supported by usual filtration aids such as cellulose, silicagel, kieselguhr, wood flour.

After completion of the filtration usually light-colored terephthalic acid esters are obtained that also satisfy the remaining specifications such as water content, residual acidity, residual content of catalyst components and residual content of monoester. For the production of light-colored terephthalic acid esters according to the method according to the invention linear or branched aliphatic mono-alcohols are used in the molecule.

The method according to the invention can be carried out continuously or batchwise within the reaction equipment typical used in chemical engineering. Agitated tanks or reaction tubes have proven appropriate, wherein the batchwise reaction management is preferred.

The synthesis steps to be used according to the invention, which are mentioned above and claimed and described in the embodiments do not underlie particular exceptional conditions with respect to their technical concept such that the selection criteria known in this field of application can be applied without restriction.

The individual combinations of components and features of the embodiments mentioned above are exemplary, the replacement and substitution of these teachings with other teachings that are included in this document with the documents cited are also explicitly contemplated. Those skilled in the art will recognize that variations, modifications and other embodiments different from those described herein may also occur without departing from the spirit and scope of the invention. Accordingly, the above description should be considered as exemplary and not as limiting. The word “comprise” used in the claims does not exclude other elements or steps. The indefinite article “a” does not exclude the meaning of a plural. The mere fact that certain amounts are recited in mutually different claims does not mean that a combination of these amounts can not be used to advantage. The scope of the invention is defined in the following claims and the associated equivalents.

1. Method of producing terephthalic acid and derivatives, comprising the steps of
a) fermentative preparation of isobutene;
b) conversion of isobutene into p-xylene;
c) oxidation into terephthalic acid; and
d) conversion into terephthalic acid derivatives.

2. Method according to claim 1, wherein between steps a) and b) no purification of the isobutene is conducted.

3. Method according to claim 1, wherein the isobutene in step a) is derived from trisaccharides, disaccharides, monosaccharides, acetone or mixtures thereof.

4. Method according to claim 1, wherein renewable raw materials are used for the fermentative production of isobutene.

5. Method according to claim 1, wherein the fermentation process is carried out at temperatures of ≧20° C. to ≦45° C. and under atmospheric pressure, and isobutene is released as a gaseous product.

6. Method according to claim 1, wherein the fermentation process is carried out at temperatures of ≧20° C. to ≦45° C. and under a pressure between 1 to 30 bar.

7. Method according to claim 1, wherein step b) comprises a cyclodimerization reaction of isobutene into p-xylene.

8. Method according to claim 1, wherein step b) comprises the following steps
b1) dimerization of isobutene into diisobutene;
b1 (i)) purification of diisobutene;
b2) conversion of diisobutylene into p-xylene.

9. Method according to claim 8, wherein step b1) is carried out under acid catalysis.

10. Method according to claim 8 wherein step b1 (i)) is carried out by distillation.

11. Method according to claim 1, wherein step c) is carried out through a liquid-phase oxidation with oxygen or atmospheric oxygen as an oxidizing agent.

12. Method according to claim 1, wherein the terephthalic acid produced according to step c) is reacted with aliphatic mono-alcohols having 1 to 11 carbon atoms.

13. Method according to claim 12, wherein the aliphatic mono-alcohols used are derived from sources of renewable raw materials.

14. Method according to claim 13, wherein the carbon amount derived from sources of renewable raw materials in the aliphatic mono-alcohols used is in the range of 0 to 100%.

15. Method according to claim 1, characterized in that bis-2-ethylhexylterephthalate is prepared.

16. Method according to claim 2, wherein the isobutene in step a) is derived from trisaccharides, disaccharides, monosaccharides, acetone or mixtures thereof.

17. Method according to claim 2, wherein renewable raw materials are used for the fermentative production of isobutene.

18. Method according to claim 2, wherein the fermentation process is carried out at temperatures of ≧20° C. to ≦45° C. and under atmospheric pressure, and isobutene is released as a gaseous product.

19. Method according to claim 2, wherein the fermentation process is carried out at temperatures of ≧20° C. to ≦45° C. and under a pressure between 1 to 30 bar.

20. Method according to claim 2, wherein step b) comprises a cyclodimerization reaction of isobutene into p-xylene.
